(19) United States
(12) Patent Application Publication
(10) Pub. No.: US 2010/0068777 A1
(43) Pub. Date: Mar. 18, 2010

(54) METHODS FOR PRODUCING BIOFUELS AND COMPOSITIONS FOR USE IN BIOFUEL PRODUCTION METHODS

(75) Inventor: Justin Reber, Highland, UT (US)

Correspondence Address: TRASKBRITT, P.C., P.O. BOX 2550, SALT LAKE CITY, UT, 84110, US

(73) Assignee: OmniLytics, Incorporated, Salt Lake City, UT (US)

(21) Appl. No.: 12/212,564

(22) Filed: Sep. 17, 2008

(51) Int. Cl. C12P 7/14; C12N 1/20; C12N 9/00

(52) U.S. Cl. 435/162; 435/252.4; 435/183

(57) ABSTRACT

Beneficial virus, such as phage, is used in fermentation methods and other techniques for producing biofuels. The beneficial virus (e.g., phage) may be targeted toward microorganisms (e.g., bacteria, etc.) that undesirably affect the fermentation or biofuel production process. Beneficial virus targeted toward microorganisms that undesirably affect the product or by-product of such a process, further processing, or the results of such further processing may also be incorporated into the fermentation or biofuel production process. Compositions that include beneficial viruses, such as phage, that are useful in such processes are also disclosed.

### TECHNICAL FIELD

The present invention relates generally to methods for producing biofuels, such as ethanol, and more specifically to fermentation and biofuel production methods in which contamination by undesirable microorganisms is minimized. More particularly, the present invention relates to the use of beneficial or useful viruses, such as phage, in fermentation and biofuel production processes. The present invention also relates to phage-containing compositions that may be used in fermentation and biofuel production processes to reduce growth, proliferation, and/or activity of microorganisms, such as bacteria, that may be undesirable in such processes, the products of such processes, the by-products of such processes, subsequent processing of the by-products, and/or the results of such subsequent processing.

### BACKGROUND OF RELATED ART

A variety of biofuel production processes are currently in development or use. Currently, the most prevalent biofuel production process is the large-scale production of ethanol. Ethanol is typically formed by fermentation processes, in which yeast digest certain “foods,” such as corn, switchgrass, or the like.

Unfortunately, a variety of microorganisms, including bacteria, are known to decrease the efficiency with which yeast produce alcohol. Bacteria may decrease the fermentation efficiency of yeast in a number of ways. For example, bacteria may produce organic acids that hinder the growth and reproduction of yeast, as well as the ability of yeast to effectively ferment sugars. Some bacteria may also infect and kill the yeast that are responsible for producing ethanol.

Conventionally, antibiotics have been used to control bacterial populations during fermentation processes that employ yeast. The overuse of antibiotics, as well as their ever-decreasing effectiveness in controlling bacterial populations, are well known and of serious concern.

### SUMMARY

As used herein, “beneficial virus” includes viruses that infect undesirable microorganisms, such as bacteria or other microorganisms that reduce process efficiencies or that impart a product with undesired characteristics. “Beneficial viruses” include bacteriophage or, more simply, “phage.” “Beneficial viruses” may also include viruses that infect other types of undesirable microorganisms, such as, but not limited to, molds, yeasts, and fungi.

In one aspect, the present invention includes the use of beneficial viruses in fermentation or other biofuel production processes. Phage and, optionally, other beneficial viruses are useful in processes by which yeast are used to ferment sugars to produce ethanol, and may also be useful in fermentation processes that are effected with bacteria (e.g., Zymomonas mobilis, etc.), as well as in other types of processes for producing biofuels, including, but not limited to, the use of algae to product biodiesel, and the use of algae to product ethanol.

The present invention also includes compositions that are formulated to minimize or eliminate undesired bacteria from biofuel production processes and/or from fermentation processes. Such a composition may, in some embodiments, be useful in ethanol production processes. Embodiments of compositions that are useful in ethanol production processes include phage or other beneficial viruses that will infect and inhibit growth, proliferation, and/or the undesirable activity of microorganisms (e.g., bacteria, etc.) that inhibit the ability of fermenting organisms (e.g., yeast, etc.) to ferment sugars into ethanol.

In other aspects, the present invention includes use of phage or other beneficial viruses to control the growth, proliferation, and/or activity of microorganisms that may be undesirable in biofuels, the by-products of fermentation or biofuel production processes, subsequent processing of the by-products, or the results of such subsequent processing. More specifically, such phage or other beneficial viruses may be incorporated into the fermentation or biofuel production process, or into the subsequent processing of by-products.

A phage-containing composition that is useful for controlling the growth, proliferation, and/or activity of microorganisms that may be undesirable in the product, by-product, subsequent processing, or result of the subsequent processing may include phage that are targeted to one or more of the undesirable microorganisms.

Other aspects, as well as various features and advantages, of the present invention, will become apparent to those in the art through consideration of the ensuing description, the accompanying drawings, and the appended claims.

### BRIEF DESCRIPTION OF THE DRAWINGS

In the drawings:

FIG. 1 is a flow chart depicting an embodiment of a biofuel production method of the present invention; and

FIG. 2 is a flow chart illustrating another embodiment of a biofuel production method according to the present invention.

### DETAILED DESCRIPTION

A composition that incorporates teachings of the present invention includes one or more types of phage or other beneficial viruses (e.g., viruses that target microorganisms, such as molds or yeasts). The beneficial viruses of the composition may target and inhibit the growth, proliferation, and/or activity of one or more microorganisms that may undesirably interfere with processes with which the composition is to be used. Alternatively, or in addition, the beneficial viruses may target and inhibit the growth, proliferation, and/or activity of one or more types of microorganisms whose presence may be undesirable in a product or by-product of the fermentation or biofuel production process, subsequent processing of a by-product, and/or any additional products resulting from subsequent processing.

In addition to including a beneficial virus, a composition of the present invention may include stabilizers or other media that support the phage, host microorganisms (e.g., bacteria when the beneficial virus is phage, etc.), and/or nutrient media for the host microorganisms.

When a composition according to the present invention includes phage, one or more of the types of phage that are included in the composition may comprise a so-called “h-mutant” phage, which includes a host range that has been broadened to enable the phage to infect a wild-type strain of a targeted microorganism, as well as one or more phage-resistant strains of the targeted microorganism. An example of a process for broadening a phage's host range is disclosed in U.S. Patent Application Publication US-2006-0153811-A1 of Jackson, et al., the entire disclosure of which is, by this reference, hereby incorporated herein.

The phage or other beneficial virus of some embodiments of the composition may consist essentially of lytic phage. In other embodiments, a composition of the present invention may include at least some lysogenic virus, provided that the beneficial virus inhibit activity of an infected host.

In embodiments of phage-containing compositions that are to be used in fermentation processes, the phage may target bacteria (or other microorganisms) that reduce the efficiency of such processes. A specific embodiment of a composition according to the present invention includes one or more types of phage that target bacteria that may be detrimental to fermentation processes. Examples of such bacteria include, but are not limited to any of a variety of species of the genus Lactobacillus (e.g., L. buchneri, L. plantarum, L. casei alactosus, L. casei casei, L. brevis 2, L. brevis 3, L. acidophilus, L. fermentum, L. lindnerii, etc.), the species Leuconostoc mesenteroides, Streptococcus equinus, Pediococcus pentosaceus, Bacillus pumilus, Bacillus cereus, and/or Clostridium butyricum, as well as various other species of bacteria. The phage that is included in such a composition may be obtained from a variety of sources, including, without limitation, bacterially contaminated fermentation mixtures, by-products of fermentation processes, and the like.

Embodiments of compositions that are to be used with other processes for producing biofuels are also within the scope of the present invention. Such compositions include one or more types of phage that will inhibit the growth, proliferation, and/or activity of bacteria or other microorganisms that may be detrimental to such processes or bacteria or other microorganisms whose inclusion in the products of such processes may be undesirable.

Another embodiment of a containing composition according to the present invention includes phage or other beneficial viruses that target bacteria or other microorganisms that are undesirable in by-products of biofuel production or fermentation processes, in the further processing of by-products, or in products resulting from such further processing. One specific embodiment of such a composition includes phage that target microorganisms (e.g., bacteria, etc.) that are undesirable in animal feeds. Such a composition may include, but is not limited to including, phage that are targeted to one of various species of the genus Campylobacter, various species of the genus Clostridium, various species of the genus Salmonella (including non-typhi serotypes of Salmonella enterica), various species of the genus Listeria, and various serotypes of E. coli. These types of phage may be obtained in a manner known in the art, from sources such as, but not limited to, the by-products of fermentation processes, the intermediates and products of feed manufacturing processes, or any other suitable source.

Other embodiments of phage-containing compositions that are useful with various by-products of fermentation or other biofuel production processes are, of course, also within the scope of the present invention.

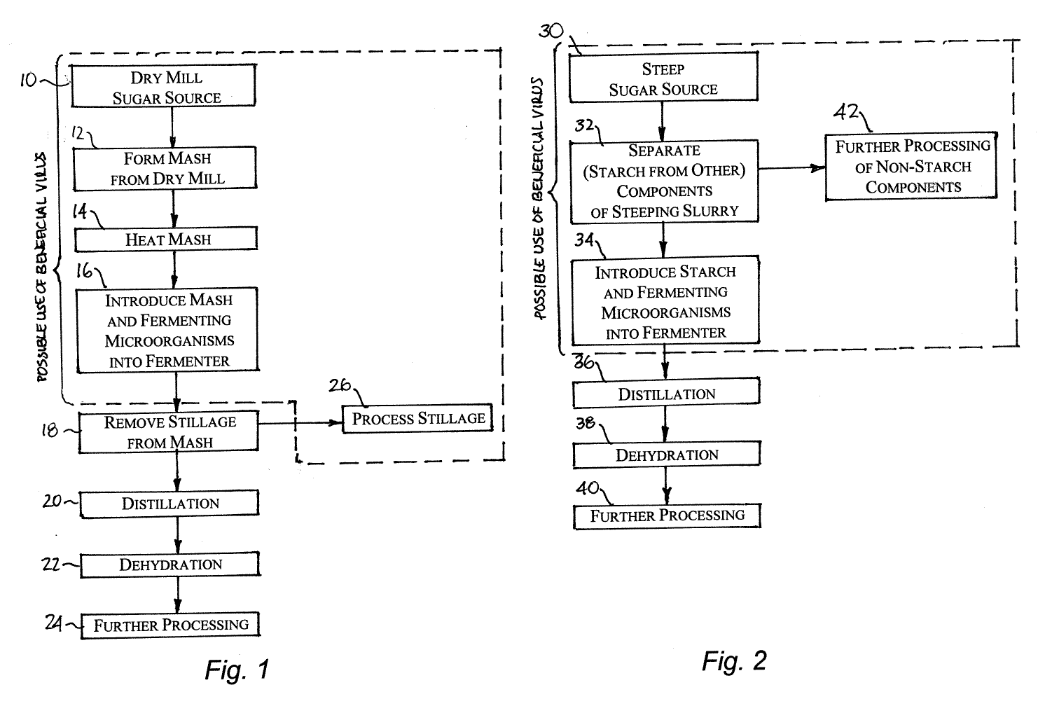

With reference to FIG. 1, an embodiment of an ethanol production process that employs so-called “dry milling” processes, in which all parts of the source of sugar are used in fermentation, is described. At reference 10, the source of sugar is dry milled to form meal. The source of sugar may comprise a grain, such as corn or rice; a grass, such as sugar cane or switchgrass; or other suitable agricultural products.

A water-based, or aqueous, liquid, which may include enzymes, may be added to the meal to form a slurry, or “mash,” at reference 12. The enzymes that are included in the mash convert complex starch molecules into simpler sugars, such as dextrose. The liquid that is used to form the mash may also include ammonia, which imparts the mash with a desired pH, as well as serve as a nutrient for the fermenting organism that will subsequently be added to the mash.

Optionally, at reference 14, the mash may be heated to reduce bacterial levels prior to fermentation. Of course, the need for heating may be eliminated by the use of phage or other beneficial viruses in the ethanol fermentation process. In embodiments where heating is not employed, time (e.g., heating time, subsequent cooling time, etc.) may be saved and other process costs (e.g., the cost of energy required for heating, the cost of heating equipment, the cost of labor associated with heating processes, etc.) may be reduced or eliminated. In embodiments where the mash is heated to reduce bacterial levels, the mash is subsequently cooled.

Next, at reference 16, the mash and a suitable fermenting organism (e.g., yeast, such as Saccharomyces cerevisiae; bacteria, such as Zymomonas mobilis; etc.) are introduced into a fermenter. Nutrients for the fermenting organism may also introduced into the fermenter. The fermenter provides the anaerobic environment that is necessary to enable the fermenting organism to convert sugar in the mash to ethanol and carbon dioxide. During fermentation, the mash may be agitated.

Phage or other beneficial viruses that control (e.g., minimizes, eliminates, etc.) growth, populations, and/or the activity of undesirable bacteria or other microorganisms in the mash during fermentation processes may be included in the mash. In some embodiments, phage or other beneficial viruses that are useful with by-products of the fermentation process may also be included in the mash. The phage or other beneficial virus may be added to the meal or the mash alone or as part of a composition, which may include other ingredients. As has already been implied, the beneficial virus or a composition containing the same may be mixed with the meal or mash at one or more parts of the foregoing process.

In some embodiments, beneficial virus or a composition that includes the same may be mixed, in dry form, with the source of sugar before the milling process, or with the meal during or after the milling process.

In other embodiments, the beneficial virus or composition may be added to the mash in wet or dry form. For example, the liquid that is used to form the mash may comprise beneficial virus. As another example, beneficial virus may be blended into the mash after the mash has been formed.

In a further embodiment, beneficial virus may be introduced into a fermenter with the mash and the fermenting organism. The addition of the beneficial virus may be effected before fermentation begins and/or during the fermentation process. Agitation of the mash during fermentation not only enhances activity by the fermenting organism, it substantially homogeneously distributes the beneficial virus throughout the mash.

Once fermentation is complete, solids, or “stillage,” may be removed from the mash and the remaining liquid may be treated by conventional distillation and dehydration processes, as represented by reference characters 18, 20, and 22, respectively, to complete the ethanol production process; i.e., to produce anhydrous ethanol.

Further processing may be conducted to manufacture a particular type of finished product, as depicted by reference character 24. In a specific embodiment, in which the anyhydrous ethanol is to be used as a fuel, an additive that renders the anhydrous ethanol undrinkable, such as gasoline or another denaturant, may be blended with the anhydrous ethanol. As a more specific example, the finished product may include about 5%, by volume, of such an additive.

In addition to producing ethanol, one or more by-products result from ethanol production processes. When ethanol production involves the use of dry milling, “stillage” (i.e., solids from the mash) remains after distillation (reference character 18). The stillage may be further processed, as indicated by reference character 26.

The presence of certain microorganisms (e.g., bacteria, etc.) within the stillage or products that are formed with or from the stillage may be undesirable. In some cases, the microorganisms that are not wanted in the stillage may differ from microorganisms that could adversely affect fermentation. Beneficial viruses (e.g., phage, etc.) that will control or eliminate microorganisms that are not wanted in the stillage may be added to the mash, as described previously, it may be added to the stillage, or it may be added to a product formed with or from the stillage.

A specific embodiment of the further use of stillage from ethanol production processes includes the manufacture of animal feed from stillage. Bacteria or other microorganism that would be undesirable in animal feed include bacteria that might be harmful to the types of animals for which the feed is intended, as well as bacteria that could spoil the feed and bacteria that could render the feed undesirable to the animals for which it is intended. Nonlimiting examples of such bacteria include various species of the genus Campylobacter, various species of the genus Clostridium, various species of the genus Salmonella (including non-typhi serotypes of Salmonella enterica), various species of the genus Listeria, and various serotypes of E. coli.

Animal feed may be formed by centrifugation of the stillage to separate solids (e.g., coarse grain) from liquids and materials that are dissolved in or otherwise carried by the liquids. Following separation, the liquid may be concentrated by known processes, such as evaporation, to a desired solids content (e.g., about 30% solids). The resulting product is known in the art as “condensed distillers solubles” (CDS), or “syrup.” The syrup is then mixed back into the solids (e.g., coarse grain) and these components are dried together. The resulting product is known in the art as “dried distillers grains with solubles” (DDGS), which may be used as a nutritious feed for livestock.

When the stillage is further processed to produce animal feed, beneficial virus or a composition that includes the same may be mixed with the stillage, the solids, the liquid, and/or when the syrup and solids are mixed together. The beneficial virus that is used in producing the animal feed may, in some embodiments, include one or more types of phage (e.g., lytic phage, etc.) and, optionally, one or more types of h-mutant phage, specific for at least one of for one or more types of undesirable bacteria that may contaminate the animal feed.

Another embodiment of ethanol production process that incorporates teachings of the present invention is a so-called “wet milling process,” which is useful when the source of sugar for fermentation is a grain. An example of an embodiment of ethanol production process that employs wet milling is illustrated by and described in reference to FIG. 2.

At reference character 30, a source of sugar is soaked, or “steeped,” in water and dilute acid (e.g., sulfurous acid, H₂SO₃) to separate the source of sugar (e.g., grain, grass, etc.) into various parts. Soaking, or steeping, of the source of sugar may, in some embodiments, be effected for about 24 hours to about 48 hours.

Next, at reference character 32, the slurry that results from soaking, or steeping, may be processed in a manner known in the art to separate the various parts of the sugar source (e.g., grain) from each other. When the sugar source is a grain, the germ may be separated from the remainder of the slurry by way of grinders. The fiber, gluten, and starch, which remain in the slurry, may then be separated from each other, as well as from the so-called “steeping liquor,” by known processes, such as centrifugation, screening processes, or use of hydrocolonic separators.

The starch may be fermented, at reference character 34. In order to ferment the starch, it is introduced, along with water (e.g., water left-over from the wet milling process, additional water, etc.) and a suitable fermenting organism into one or more fermenters. Nutrients for the fermenting organism may also introduced into the fermenter. During fermentation, the mixture of starch and fermenting organism may be agitated.

A beneficial virus, in any suitable form known in the art, may be included in the fermentation mixture to control (e.g., minimize, eliminate, etc.) the growth, population, and/or activity of one or more types of microorganisms (e.g., bacteria, etc.) that may have an adverse effect on fermentation. The beneficial virus may be added to the source of sugar before or during the wet milling process, to the slurry obtained from the wet milling process, to the starch that is obtained from the slurry, and/or to the fermentation mixture.

Once fermentation is complete, conventional distillation and dehydration processes may be effected, as represented by reference characters 36 and 38, respectively, to complete the ethanol production process; i.e., to produce anhydrous ethanol.

In some embodiments, further processing, at reference character 40, is necessary to form a particular type of finished product. For example, when the anhydrous ethanol is used as a fuel or as part of a fuel, an additive that renders the anhydrous alcohol undrinkable, such as gasoline or another denaturant, may be blended with the anhydrous ethanol. As a more specific example, the finished product may include about 5%, by volume, of such an additive.

As with ethanol production processes that employ dry milling, there are by-products of ethanol production processes that utilize wet milling techniques. One or more types of beneficial viruses may be useful in processing some of these by-products. For example, in some embodiments, the steeping liquor may be concentrated (e.g., by evaporation). The resulting “heavy steep water” may be re-blended with the fiber to manufacture gluten feed for livestock. Beneficial viruses (e.g., phage, etc.) that will control the growth of undesired microorganisms (e.g., bacteria, etc., including harmful bacteria, bacteria that may spoil the feed, bacteria that may render the feed undesirable to the animals for which it is intended, etc.) may be introduced into the steep liquor, the heavy steep water, the fiber, and/or the mixture of heavy steep water and fiber, or in any other manner that will incorporate beneficial virus into the gluten feed in its finished form.

In other embodiments, beneficial virus (e.g., phage, etc.) may be introduced into the gluten (protein), which may be filtered and dried by known processes to form gluten meal, which is used to feed broiler poultry (i.e., poultry that are to be used as food). The phage that is introduced may be targeted toward and control populations of one or more types of microorganisms (e.g., bacteria, etc.) that may be harmful to the poultry, that may spoil the gluten meal, and/or that may render the gluten meal undesirable to the poultry. The beneficial virus may be introduced into the gluten before or after the gluten is filtered.

Although the foregoing description contains many specifics, these should not be construed as limiting the scope of the present invention, but merely as providing illustrations of some exemplary embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions, and modifications to the invention, as disclosed herein, which fall within the meaning and scope of the claims are to be embraced thereby.

What is claimed:

1. A process for producing ethanol, comprising:
milling a source of sugar;
forming a fermentation mixture including at least a portion of the source of sugar and a fermenting organism;
introducing beneficial virus into the presence of the source of sugar; and
fermenting the sugar in the presence of the beneficial virus to control a population of at least one type of undesirable microorganism during fermentation.

2. The process of claim 1, wherein milling the source of sugar comprises milling at least one of a grain and a grass.

3. The process of claim 1, wherein milling the source of sugar comprises milling corn.

4. The process of claim 1, wherein forming the fermentation mixture comprises forming the fermentation mixture to include Saccharomyces cerevisiae.

5. The process of claim 1, wherein forming the fermentation mixture includes forming the fermentation mixture to further include at least one enzyme for converting starch to simpler sugars.

6. The process of claim 5, wherein forming the fermentation mixture includes forming the fermentation mixture to further include ammonia.

7. The process of claim 1, wherein introducing beneficial virus comprises introducing beneficial virus specific for at least one microorganism that inhibits fermentation of the sugar by the fermenting organism.

8. The process of claim 7, wherein introducing beneficial virus comprises introducing beneficial virus specific for at least one microorganism that may undesirably affect a by-product of the process or processing of the by-product.

9. The process of claim 7, wherein introducing beneficial virus comprises introducing lytic beneficial virus.

10. The process of claim 7, wherein introducing beneficial virus consists of introducing lytic beneficial virus.

11. The process of claim 1, wherein introducing beneficial virus is effected before milling, during milling, after milling, while forming the fermentation mixture, or after forming the fermentation mixture.

12. The process of claim 1, wherein introducing beneficial virus comprises introducing bacteriophage into the presence of the source of sugar.

13. A fermentation mixture for producing ethanol, comprising:
a source of sugar;
at least one fermenting organism; and
beneficial virus for controlling at least one of growth, a population, and activity of at least one microorganism during fermentation of the sugar.

14. The fermentation mixture of claim 13, wherein the at least one fermenting organism comprises Saccharomyces cerevisiae.

15. The fermentation mixture of claim 13, wherein the beneficial virus comprises lytic virus.

16. The fermentation mixture of claim 13, wherein all of the beneficial virus included in the fermentation mixture consists of lytic virus.

17. The fermentation mixture of claim 13, further comprising:
at least one enzyme for converting starch of the source of sugar to simpler sugars.

18. The fermentation mixture of claim 13, further comprising:
ammonia.

19. The fermentation mixture of claim 13, further comprising:
beneficial virus for controlling at least one of growth, a population, and activity of at least one microorganism that undesirably affects a by-product of a fermentation process or processing of the by-product.

20. The fermentation mixture of claim 13, wherein the beneficial virus comprises bacteriophage.

21. A biofuel production process incorporating beneficial virus to prevent at least one of growth, proliferation, and activity of at least one microorganism that undesirably affects the biofuel production process, a product of the biofuel production process, a by-product of the biofuel production process, or processing of the by-product.

22. The biofuel production process of claim 21, wherein incorporating beneficial virus comprises incorporating beneficial virus into a fermentation process.
